(19) United States
(12) Patent Application Publication
(10) Pub. No.: US 2006/0134185 A1
(43) Pub. Date: Jun. 22, 2006

(54) Self-adhesive reabsorbable hemostyptic

(76) Inventors: Erich K. Odermatt, Schaffhausen (CH); Jurgen Wegmann, Wurmlingen (DE); Bernd Blender, Hohentengen (DE); Jurgen Duffner, Radolfzell (DE)

Correspondence Address: NATH & ASSOCIATES, 112 South West Street, Alexandria, VA, 22314, US

(21) Appl. No.: 10/552,963

(22) PCT Filed: Apr. 13, 2004
(86) PCT No.: PCT/EP04/03850

§ 371 Date: Nov. 29, 2005

(30) Foreign Application Priority Data
Apr. 17, 2003 (DE) 10318802.9

(51) Int. Cl. A61K 9/70

(52) U.S. Cl. 424/443

(57) ABSTRACT

The invention relates to a reabsorbable hemostyptic self-adhering to human or animal tissue and essentially consisting of at least one polymer which carries free aldehyde groups and whose aldehyde groups are able to react with nucleophilic groups of the tissue, the hemostyptic being present in solid, in particular dry, porous and absorbent form, to a method for its production, and to the provision of the hemostyptic according to the invention for diverse medical indications.

The invention relates to a reabsorbable hemostyptic self-adhering to human or animal tissue and essentially consisting of at least one polymer which carries free aldehyde groups and whose aldehyde groups are able to react with nucleophilic groups of the tissue, the hemostyptic being present in solid, dry, porous and absorbent form, to a method for its production, and to the provision of the hemostyptic according to the invention for diverse medical indications.

In modern surgery, tissue adhesives, such as those described in U.S. Pat. No. 6,156,488 or DE 101 52 407, for example, are increasingly being used for closing internal and external wounds in body tissue and for joining together two separated tissue parts, especially in minimally invasive surgery. However, these liquid tissue adhesives cause difficulties in certain applications, for example in the adhesion of a planar wound, in the repair of curved tissue parts or in cases of difficultly accessible wounds, and in the closure of internal wounds in minimally invasive surgical procedures, the reason being that the liquid tissue adhesives cannot be applied uniformly at every desired site of the wounds to be closed or of the tissue parts to be joined together.

It is also known to use collagen nonwovens for hemostasis of internal wounds. Nonwoven hemostyptics of this kind are sold, for example, under the trade name TachoComb H by Nycomed and under the trade name Lyostypt by B. Braun-Melsungen A G. Compared to the liquid tissue adhesives, these nonwoven hemostyptics have the advantage of being able to be applied uniformly across the wound surface, even in the case of internal wounds with irregular surfaces. To ensure that they remain on the wound and to prevent their slipping or falling off, in the case of the TachoComb H collagen nonwoven fabric a two-component adhesive is added to the nonwoven material at the time of production, in order to ensure a tissue adhesion action of the hemostatic nonwoven on the tissue surface. Hemostatic collagen nonwovens of this kind, without a tissue adhesive component in most cases based on fibrin adhesives, have only a very low adherence to hemorrhaging tissue surfaces. The adhesive components mostly consist of thrombin and fibrinogen, said thrombin and fibrinogen intervening actively in the blood coagulation cascade. A serious disadvantage of the hemostyptics with collagen or with thrombin and fibrinogen is to be seen in the animal origin, in most cases bovine, porcine or equine origin, or human origin, of the collagen, thrombin and fibrinogen. Against the background of the problems of BSE and HIV in particular, a risk of infection through use of surgical material of animal or human origin can no longer be excluded. Moreover, in the presently available hemostyptic nonwovens of animal origin, it is necessary to add an adhesion-promoting component in the form of in most cases a two-component adhesive, which has to be incorporated into the nonwoven. These adhesive components in the form of thrombin and fibrinogen, however, constitute an intervention in the blood coagulation cascade.

European patent application EP 815 879 from Johnson & Johnson Medical Incorporation describes a bioabsorbable material which, from oxidized polysaccharides, can be used in the form of a freeze-dried sponge for hemostasis and for avoiding adhesion in surgical interventions. The bioabsorbable material consists of a water-soluble cellulose derivative which has primary alcohol groups in the range of 3 to 12% oxidized to the carboxylic acid. This bioabsorbable material, sold commercially under the trade name TABOTAMP, is primarily used for prevention of hemorrhages after surgical interventions and, in line with its underlying purpose, therefore has no adhesive properties whatsoever in respect of tissue or tissue parts.

EP 693 291 from the United States Surgical Corporation, USA, discloses a wound composition in the form of a powder or in combination with a delivery vehicle in the form of a liquid or paste from an oxidized cross-linked polysaccharide for treatment of a wound site. Wound treatment compositions of this kind are also known under the commercial name Debrisan for absorption of wound exudates and for removal of foreign bodies from tissue wounds. The oxidized, cross-linked polysaccharide used in EP 693 291 has monosaccharide hydroxyl groups oxidized to the carboxylic acid. Like the liquid tissue adhesives too, this composition also poses the problem of uniform application to uneven and curved wounds or tissues or tissue parts. Uniform coverage of a wound is not possible with a powder or liquid composition of this type. In addition, for closure of the tissue wound after treatment with the powdered wound treatment composition, wound closure by another auxiliary in the form of a wound closure band is also necessary, since the composition itself has no adhesive properties at all.

It is therefore desirable to make available a hemostyptic of non-animal or non-human origin which can be applied uniformly irrespective of the nature and surface of the wound and which does not require any additional auxiliaries for fixing to the wound or for closure of the wound, and also to make available a method for production of this hemostyptic.

According to the invention, this object is achieved by a reabsorbable hemostyptic self-adhering to human or animal tissue and essentially consisting of at least one polymer which carries free aldehyde groups and whose aldehyde groups are able to react with the nucleophilic groups of the tissue, the hemostyptic being present in solid, porous and absorbent form, in accordance with independent claim 1. Advantageous developments are described in claims 2 through 17.

A further solution lies in a method for producing the hemostyptic, in accordance with independent claim 18.

The object is likewise achieved through the provision of a hemostyptic in accordance with independent claims 19 through 23.

The wording of all the claims is incorporated by reference into the content of the description.

The hemostyptic according to the invention is generally present in dry form. However, it can also be present in moist form. It advantageously has a fibrous structure and is in particular present as a nonwoven,

An advantage of the hemostyptic according to the invention is that it adheres to the wound surface without additional fixing agents or adhesive components and, by virtue of its porous and absorbent form, permits rapid closure of a heavily bleeding wound. As soon as it has been applied to the wound, the hemostyptic can no longer be moved. A further advantage lies in the preferably solid and nonetheless elastically compressible form of the hemostyptic which, even in minimally invasive surgical procedures on difficultly accessible internal wounds, can be applied in a uniform thickness across the entire wound surface.

A further advantage lies in the non-bovine or non-human origin of the starting material of the hemostyptic according to the invention, which means that it is possible to rule out possible infection in respect of Creutzfeldt-Jakob disease and BSE and HIV problems.

The hemostyptic according to the invention consists essentially of at least one polymer which carries free aldehyde groups and whose aldehyde groups are able to react with the nucleophilic groups of the human or animal tissue, in particular amino groups, SH groups and OH groups. The hemostyptic is self-adhering to human or animal tissue, i.e. can be applied to a wound surface without additional fixing agents. It is reabsorbable, i.e. it is completely degraded in the human or animal body after a certain time.

The hemostyptic is present in solid, in particular dry form and preferably has a spongy or fibrous porous structure. The hemostyptic has strong absorption properties, resulting in a high concentration of blood platelets on the surface and in the inside of the hemostyptic.

The hemostyptic forms an adhesive connection, in particular by imune bonds, between the aldehyde groups of the polymer and the amino groups of the blood, of the serum and especially of the surrounding body tissue. These imine bonds are reversible covalent bonds which are stronger than purely ionic bonds and therefore permit good adherence between hemostyptic and tissue. In the case of SH or OH groups, the hemostyptic according to the invention forms adhesive connections in the form of acetal or thioacetal bonds which behave correspondingly to the imine bonds. By means of the hemostyptic according to the invention, not only is the blood bound at the wound site by the absorbent action, the hemostyptic also begins to form a gel and acts as a mechanical barrier which prevents the escape of blood. Moreover, because of its adhesion properties, it closes the tissue site to be repaired and, through this closure of the wound, additionally strengthens its hemostatic action and thus also prevents possible union with adjacent tissue. By means of the hemostyptic according to the invention, the blood is bound by purely physically chemical means, without intervening in the blood coagulation cascade as do thrombin and fibrinogen. The hemostyptic according to the invention is not a medicament.

In one embodiment, the hemostyptic consists only of one polymer. In another embodiment, the hemostyptic consists of a combination of different polymers. In another embodiment, the polymer has cross-linkages, via which the stability and hardness of the hemostyptic can be adjusted. The degradation time can also be adjusted or slowed down by addition of cross-linking agents. The polymer of the hemostyptic is preferably present in uncrosslinked form.

It is also conceivable that the hemostyptic contains additives, in particular softeners. In one embodiment, the hemostyptic contains glycerol as softener. It is also conceivable that the hemostyptic contains pharmacologically active substances which are released from the hemostyptic to the surrounding tissue and body fluids.

The hemostyptic can also contain an additive for increasing the absorption power. Absorption power here refers both to the speed of fluid uptake and also to the absolute quantity taken up. The time for hemostasis is additionally shortened by this means, and the risk of blood passing through the hemostyptic is reduced. In a particular embodiment, the additive used to increase the absorbency is carboxymethylcellulose (CMC).

The hemostyptic according to the invention can be elastically compressible. In one embodiment for improving elasticity, the hemostyptic is pressed in a substantially reversible manner by means of a pressing device, in particular to a thickness of 0.3-0.4 mm, after production. The flexibility of the hemostyptic is considerably increased by this pressing. The break-off angle of the hemostyptic, when bending from the plane in the dry state, is advantageously 10° for the unpressed hemostyptic and advantageously 30° for the pressed hemostyptic. In this way, modeling the hemostyptic to the wound is made very much better and easier, without the hemostyptic breaking.

In one embodiment, the hemostyptic has the form of a three-dimensional body and is preferably present in the form of a sheet.

In a particular embodiment, the hemostyptic is present in fibrous form, preferably in the form of an in particular three-dimensional nonwoven, with a fibrous structure having a total surface area which is many times greater than the surface area of the nonwoven.

It is also conceivable that the hemostyptic is present in the form of an open-cell foam. This foam has, in relation to the surface area of the hemostyptic, a many times larger inner surface.

According to further embodiments, the hemostyptic is present in the form of a granulate or a powder of absorbent particles.

In another embodiment, the hemostyptic is present in the form of a film or membrane. The film or membrane can be obtained by pressing or rolling of a foam or lyophilisate or by pouring of a solution and subsequent drying of the solution. It is also possible to produce a film by knife-coating of a dextran aldehyde solution.

The hemostyptic according to the invention can be present in various three-dimensional forms. It is conceivable that the hemostyptic is present in the form of a sheet-like patch, or in the form of a ring. It is also conceivable that the hemostyptic is tubular and elastic or dimensionally stable.

The polymer and preferably the entire hemostyptic is advantageously water-soluble. In this way, the hemostyptic according to the invention is fully dissolvable in the different aqueous body fluids. The time for it to dissolve in the body can be adjusted through the degree of cross-linking. The hemostyptic can also be present in moist form and, in special cases, in particular in the form of a solution, preferably in a single-chamber syringe, or in the form of a gel, preferably in a two-chamber syringe, and can preferably be used in this form. The particular features of the solid, dry and absorbent form are then omitted.

According to one embodiment, the polymer carrying aldehyde groups is an oxidized, in particular bioabsorbable polysaccharide. In a particular embodiment, the polysaccharide is dextran polyaldehyde. Other oxidized polysaccharides are also possible, in particular starch, agar, cellulose, xanthan, heparin, alginic acid and hyaluronic acid. Combinations of different polysaccharides with aldehyde groups are also possible.

It is also conceivable that the polymer carrying aldehyde groups is an in particular branched polyethylene glycol PEG. In this embodiment, the polyethylene glycol has at least three terminal aldehyde groups, which can form imine bonds with the amino groups of the body tissue and of the body fluids. The terminal aldehyde groups can also react in the body with SH or OH groups to give acetals or thioacetals.

In another embodiment, the polymer carrying aldehyde groups is an in particular branched polyvinyl alcohol (PVA) which has at least three terminal aldehyde groups.

In the two abovementioned embodiments in which the polymer is a PEG or PVA, the aldehyde group function within the molecule can be set apart from the polymer backbone by means of a spacer (FIG. 23). The synthesis of the spacer is shown in FIG. 22. The synthesis and binding of the spacer is described in the examples.

In further embodiments, other biocompatible polyols or polyethylene oxide (PEO) can also be used as polymer backbone.

The proportion of glucose units oxidized to the aldehyde in the dextran polyaldehyde is advantageously at least 20%, preferably 35-100%, in particular between 60 and 80%. By means of a high proportion of glucose units oxidized to the aldehyde, the multiplicity of covalent bonds, in particular imine bonds, permits strong adhesion of the hemostyptic to the body tissue.

According to one embodiment, the hemostyptic according to the invention can be obtained by lyophilization of a solution of the at least one polymer. It is also conceivable that the hemostyptic can be obtained from a solution of the at least one polymer by foaming. A much larger surface and more aerated structure of the hemostyptic can be achieved by addition of crushed ice.

According to one embodiment, the hemostyptic can be obtained from a 0.5% to 20% strength, preferably 1% to 15% strength, solution of the at least one polymer. In a particularly preferred embodiment, the hemostyptic can be obtained from a 1% to 10% strength, especially 2.5% strength solution of the at least one polymer.

Because of its sponge-like structure and porosity and its hydrophilic character, the hemostyptic according to the invention can take up at least 30 times its own weight of fluid. In a particular embodiment in which the hemostyptic is additionally pressed after production, the swelling capacity or water uptake can be reduced. This reduction of the uptake of water to a maximum of 20 times its own weight is to be compensated partially by the additive for increasing the absorbency and is always sufficient to close heavily bleeding wounds. The increased elasticity of the hemostyptic achieved by pressing, and the associated improved adaptation to the surface, again approximately equals this effect of the pressing. In addition, the hemostyptic is able to take up at least 3 times its own weight of hemoglobin.

In a further embodiment, the at least one polymer carrying aldehyde groups is partially cross-linked, before use, with a cross-linking agent, preferably chitosan. However, other cross-linking agents are also conceivable in the form of bifunctional amines, in particular the amino acids lysine, ornithine, arginine or triethylene glycol diamine, multifunctional amines, in particular the polyamino acid polylysine, bifunctional or multifunctional molecules containing SH— or NH₂— groups, in particular cysteine or polycysteine, or bifunctional or multifunctional thiols, and also peptides.

In a particular embodiment, the hemostyptic has a surface structured at least on one side. The structured surface improves the adherence of the hemostyptic to the tissue. The structuring can be applied on one side or both sides. Various types of structuring are conceivable, such as a square, jagged, braided, woven or spiral-shaped structure.

By means of the structuring, the mechanical friction between tissue and hemostyptic is increased through the enlarged surface area, and the hemostyptic, after application, holds better at the applied position. It is also possible to apply a structure in the form of perforations, in particular by punching or by pressing a needle board, to the hemostyptic. The initially closed surface is thus made more easily accessible for the fluid that is to be taken up, and the time for hemostasis and also the absorbency are generally increased.

The hemostyptic can preferably be colonized by cells after just a few days. For example, liver cells grow into the structure of the hemostyptic after just 7 days. This ensures rapid healing of the wound and restoration of complete functioning of the tissue.

The hemostyptic according to the invention is preferably present in sterilized form, in particular in a sterilized package.

The invention further relates to a method for producing a hemostyptic which involves a polymer in solution and/or in the gel state being converted by lyophilization into a solid dry form, the polymer preferably being a polysaccharide and preferably dextran polyaldehyde. The solution medium used is preferably water or an aqueous CaCl₂ solution.

A structuring can be applied either by suitably structured lyophilization dishes or by embossing at least one surface following production of the nonwoven.

The invention further relates to the provision of the hemostyptic for a preferably internal application in a human or animal organism, in particular for wounds.

In an advantageous embodiment, the invention further relates to the provision of the hemostyptic for closure of wounds, preferably of internal wounds.

Another aspect of the invention is the provision of the hemostyptic in cases of organ resection or organ rupture, particularly of the liver, kidneys, spleen or pancreas. For further possible uses, see FIG. 20.

A further aspect of the invention is the provision of the hemostyptic in the form of a ring for anastomoses.

Another aspect of the invention is the provision of the hemostyptic in the form of a nonwoven, a membrane or a film for adhesion prophylaxis or as barrier.

The present invention is explained below by detailed description of a particular embodiment and by means of figures. In this embodiment, individual features of the invention may be realized alone or in combination with other features. The particular embodiment described serves to explain and to provide a better understanding of the invention and is not to be regarded as in any way limiting the invention.

### DESCRIPTION OF THE FIGURES

FIG. 1 shows a magnified view of a dextran aldehyde nonwoven consisting of a 1% strength dextran aldehyde solution,

FIG. 2 shows a magnified view of a dextran aldehyde nonwoven consisting of a 2% strength dextran aldehyde solution,

FIG. 3 shows a magnified view of a dextran aldehyde nonwoven consisting of a 3.5% strength dextran aldehyde solution,

FIG. 4 shows a magnified view of a dextran aldehyde nonwoven consisting of a 5% strength dextran aldehyde solution,

FIG. 5 shows a magnified view of a dextran aldehyde nonwoven consisting of a 7.5% strength dextran aldehyde solution,

FIG. 6 shows the results of a Lee-White clotting test,

FIG. 7 shows a liver resection,

FIG. 8 shows the sealing after a liver resection,

FIG. 9 shows traumatization of a liver by cross incision,

FIG. 10 shows hemostasis of the traumatized liver from FIG. 9,

FIGS. 11 through 14 show the use of the hemostyptic in the form of an anastomosis ring,

FIG. 15 shows the hemostyptic in the form of a nonwoven,

FIG. 16a shows the hemostyptic in the form of an anastomosis ring,

FIG. 16b shows an enlarged detail from FIG. 19a,

FIG. 17 shows the use of a dextran aldehyde membrane,

FIG. 18 shows the synthesis of a spacer,

FIG. 19 shows the binding of a spacer,

FIG. 20 shows the possible uses of the hemostyptic in the case of an organ resection or rupture,

FIG. 21 shows a liver traumatized by cross incision,

FIG. 22 shows the shaping of a hemostyptic,

FIG. 23 shows the traumatized liver after treatment with the hemostyptic, and

FIG. 24 shows a section through the traumatized liver.

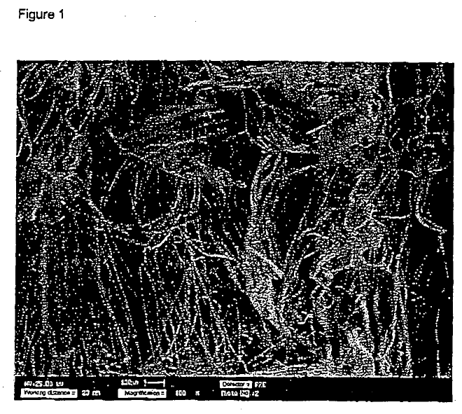

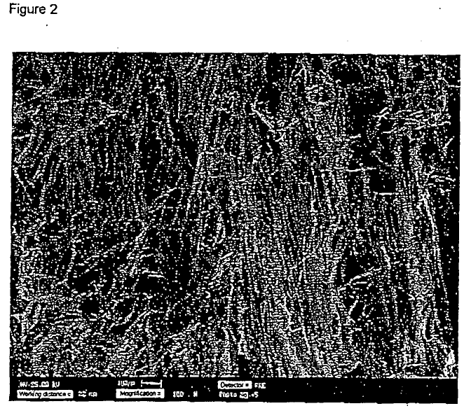

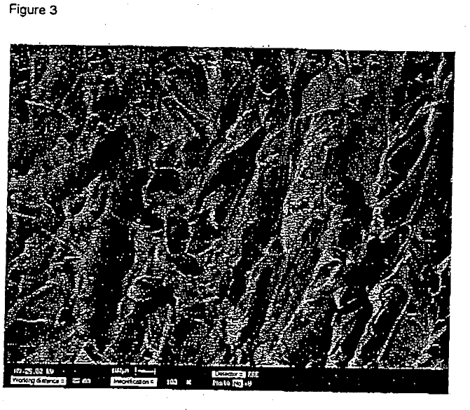

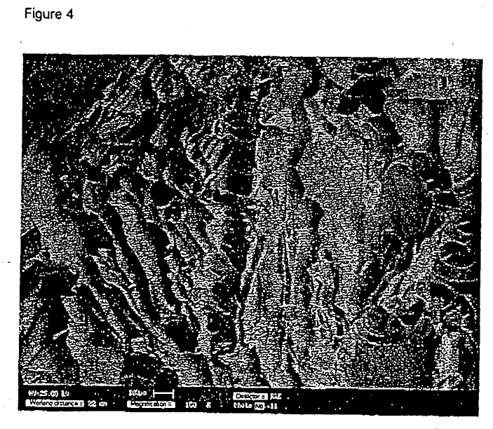

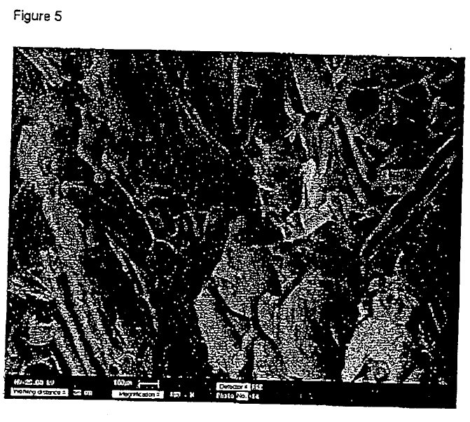

FIGS. 1 through 5 each show 100× magnifications of the dextran aldehyde nonwoven structure produced from dextran aldehyde solutions at the concentrations of 1%, 2%, 3.5%, 5% and 7.5% corresponding to nonwovens 2 through 6 in Table 1. With increasing concentration of the dextran aldehyde solution, and with the solution to be lyophilized remaining at the same filling level, the structure of the dextran aldehyde nonwoven becomes noticeably denser and shows a fibrous structure in the range from 1% to 2%. From a dextran aldehyde concentration of 3.5% through 7.5%, a spongy structure can increasingly be observed, comprising tube-shaped and cavity-forming structures.

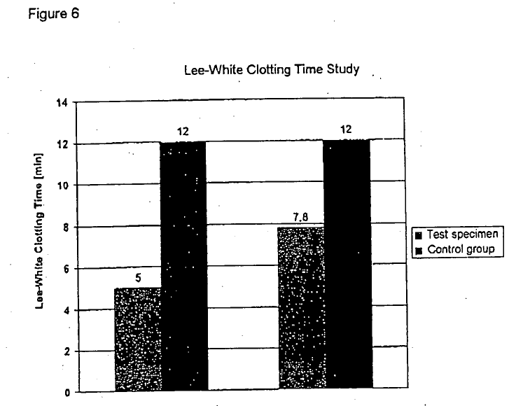

FIG. 6 shows the results of a Lee-White clotting time study for coagulation of a 15% strength aqueous dextran aldehyde solution and of a dextran aldehyde nonwoven (produced from 2% strength aqueous dextran aldehyde solution). For the dextran aldehyde solution and also for the dextran aldehyde nonwoven, the result is in each case shown for the respective test substance (hatching) and the parallel control reaction without test substance (without hatching). The Lee-White clotting time for the 15% strength dextran aldehyde solution was 5 minutes and, for the dextran aldehyde nonwoven produced from 2% strength aqueous dextran aldehyde solution, it was 7.8 minutes. The clotting times for the control reactions without test substances were in both cases 12 minutes, so that a clotting time reduction of ca. 30% was able to be obtained for the dextran aldehyde nonwoven.

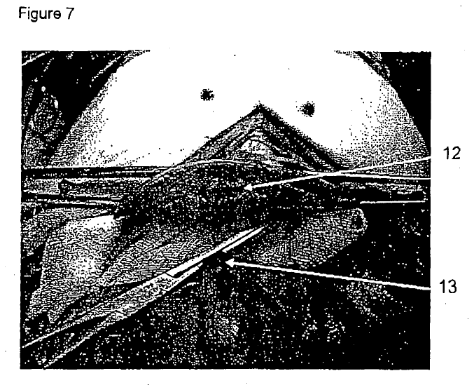

FIG. 7 shows a liver resection on a pig's liver in which the blood supply was interrupted by a Pringle maneuver and parenchyma clamp.

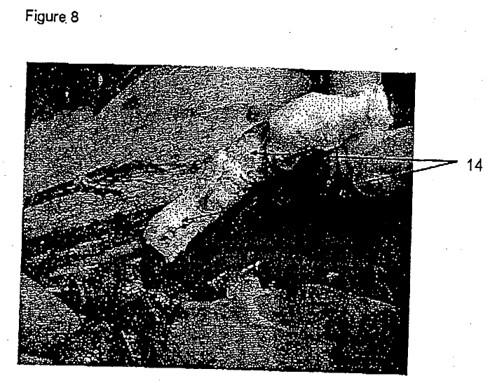

FIG. 8 shows, subsequent to this liver resection (see FIG. 7), the hepatic vessels fixed with suture material, and the vessels subsequently sealed with the polyaldehyde nonwoven.

FIG. 9 shows traumatization of a pig's liver by a cross incision measuring 2×3 cm long and 5 cm deep, with intact blood supply to the liver,

FIG. 10 shows treatment of the cross incision (see FIG. 9), with intact blood supply to the liver. By moistening a polyaldehyde nonwoven with a moistened compress in order to staunch the hepatic bleeding. The bleeding of the traumatized liver was able to be completely staunched here by the moistened nonwoven.

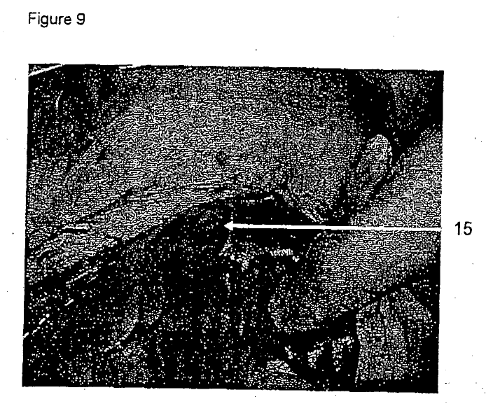

FIGS. 11 through 14 show the use of the hemostyptic as anastomosis ring. Here, the hemostyptic is present in the form of a tubular part.

FIG. 11 shows a first blood vessel portion 1 with the free end 8 which is to be connected, the tubular hemostyptic 2 being pushed onto the outer face 4 of the blood vessel portion such that it is spaced apart from the free end 8.

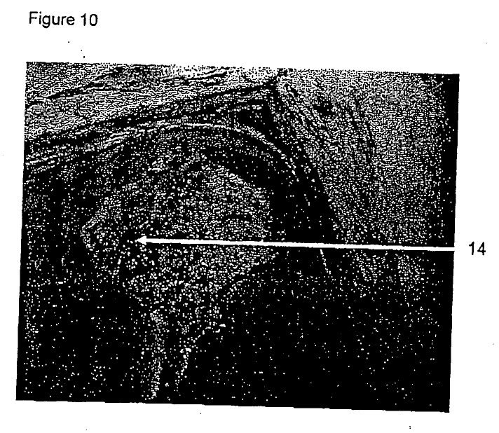

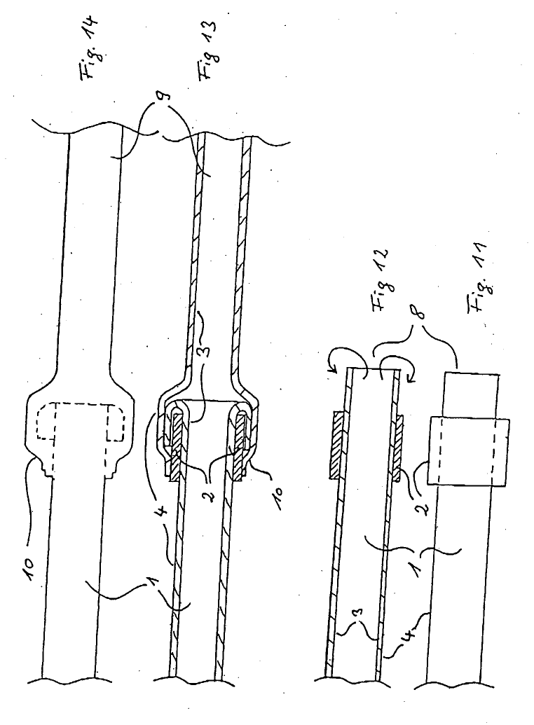

FIG. 12 shows a cross section through the first blood vessel portion 1 over which, at a spacing from the free end 8, a tubular hemostyptic 2 is pushed. The two arrows indicate how the free end 8 of the first blood vessel portion 1 is turned outward over the tubular hemostyptic 2 and, in this way, the intima/inner face 3 of the blood vessel is directed outward.

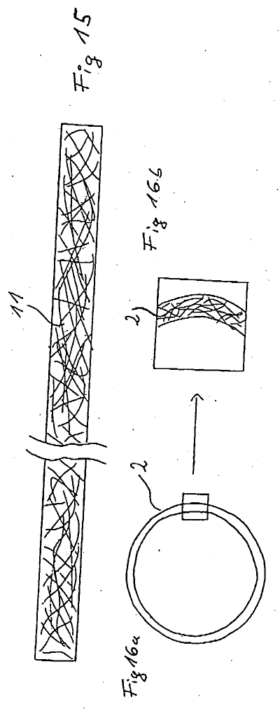

FIG. 13 and FIG. 14 show the free end 10 of the second blood vessel portion 9 turned from outside over the end of the first blood vessel 1, the two inner faces 3 of the first and second blood vessel portions 1, 9 coming to lie on one another. After the hemostyptic in the form of an anastomosis ring 2 has been pulled over the first vessel 1, as shown in FIGS. 11 and 12, the free end, as indicated by the two arrows in FIG. 12, is turned back outward over the anastomosis ring 2 so that a part of the anastomosis ring is covered by the free end 8 of the first blood vessel portion 1 and, in this way, the inner face 3 of the first blood vessel 1 is directed outward. A second blood vessel 9 is then pulled over the inverted end of the blood vessel 1, specifically to such an extent that the free end 10 of the second blood vessel portion 9 is guided over that part of the anastomosis ring 2 not yet covered by the free end 8 of the first blood vessel portion 2. The superpositioning of the first and second blood vessel portions in the overlapping area results in mutual contact between the two inner faces of the vessels, and, after the intervention, this contact leads to rapid and problem-free union of the two blood vessel portions and promotes compact formation of the intima.

FIG. 15 shows a plan view of the cross section of a three-dimensional sheet-like hemostyptic in the form of a nonwoven 11 which has been lyophilized from a dextran aldehyde solution. The plan view shows the fibrous structures of the lyophilized dextran aldehyde which fill the entire surface of the hemostyptic in an unordered fashion. The height of the nonwoven is determined here by the filling level of the dextran aldehyde solution prior to lyophilization and does not change during the course of the lyophilization of the dextran aldehyde solution. The proportion of fibrous dextran aldehyde in this plan view clearly shows the high number of hollow spaces between the fibrous dextran aldehyde structures, which provides the high degree of absorbency of the nonwoven.

FIG. 16 shows the hemostyptic in the form of an anastomosis ring whose use is illustrated in FIGS. 11 through 14. FIG. 16a shows the plan view of the cross section through the annular hemostyptic 2. FIG. 16b shows an enlarged detail from FIG. 16a, this detail making clear the fibrous dextran aldehyde structure of the anastomosis ring 2.

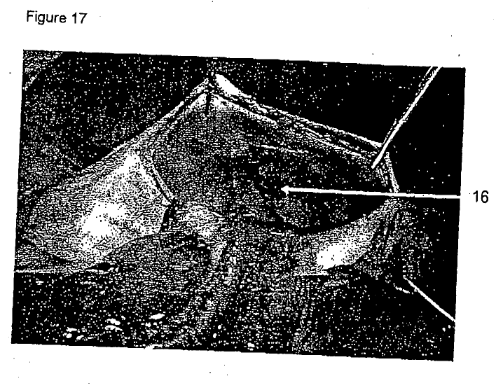

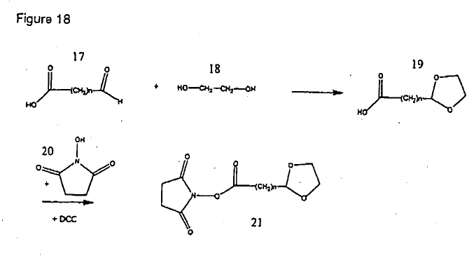

FIG. 17 shows the use of a dextran aldehyde membrane 16 as adhesion prophylaxis on a traumatized abdominal wall of a rabbit (rabbit side wall model).

FIG. 18 shows the synthesis of a spacer for insertion between the polymer and the aldehyde group function (see also example).

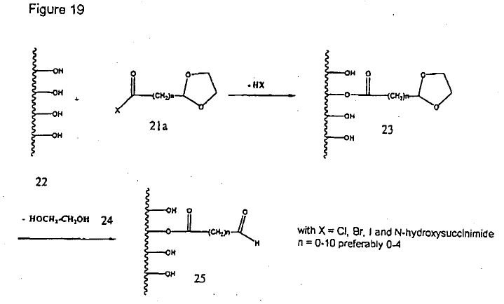

FIG. 19 shows the binding of a spacer first to the polymer, and the subsequent preparation of the aldehyde function (see example).

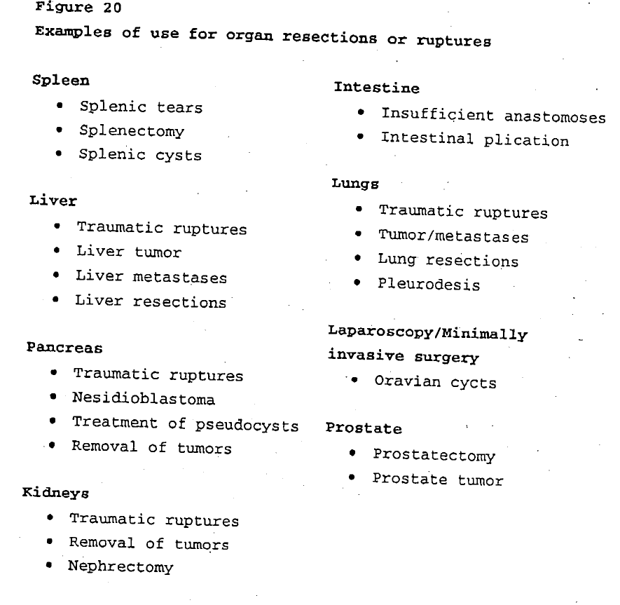

FIG. 20 shows the possible uses of the hemostyptic in the case of an organ resection or rupture.

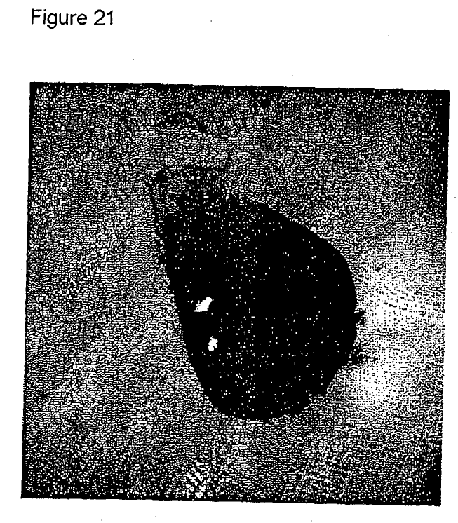

FIG. 21 shows a rat liver traumatized by cross incision.

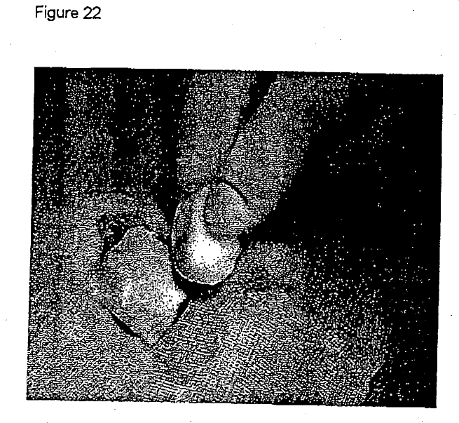

FIG. 22 shows a pressed hemostyptic according to Example 7 being modeled onto the traumatized liver from FIG. 21.

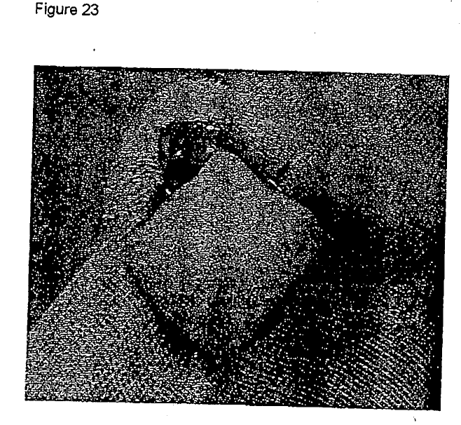

FIG. 23 shows the traumatized liver after treatment and successful hemostasis with the hemostyptic according to Example 7.

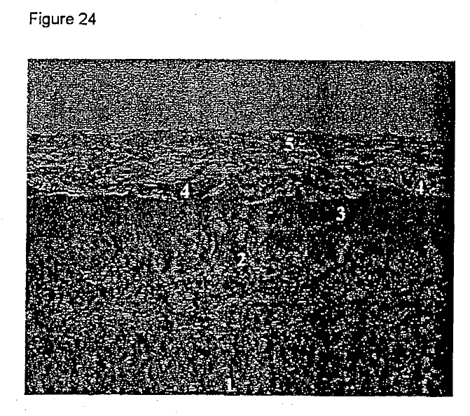

FIG. 24 shows a histopathology section through the traumatized liver from FIG. 21 which was removed 7 days after the operation. In the figure, (1) indicates liver parenchyma, (2) fibrous capsule, (3) liver regeneration tissue, (4) nonwoven material, (5) Outer connective tissue layer.

### EXAMPLES

1. Production of a Hemostyptic in Nonwoven Form:

Dextran aldehyde is dissolved in bidistilled water at 50° C. The solution is poured into dishes and lyophilized, the filling level of the dishes with the dextran aldehyde solution determining the thickness of the hemostyptic nonwoven. Nonwovens were produced from solutions of different concentration of dextran aldehyde. The weight per unit area of the nonwovens can be adjusted via the concentration and thickness of the nonwoven. No coherent nonwoven was obtained by lyophilization from the 0.5% strength dextran aldehyde solution. The lyophilisate consisted of individual fragments. With increasing concentration of dextran aldehyde, the structure of the fibrous hemostyptic nonwoven becomes denser (see FIGS. 1 through 5). With increasing concentration and density of the structure, increasingly harder and more pressure-stable nonwovens are obtained, which at the same time lose elasticity.

TABLE 1
Variation of dextran aldehyde concentration

| | | Filling | | |
|---|---|---|---|---|
| | Concentration | level | | |
| | of starting | of | Nonwoven | Weight per |
| Nonwoven | solution | dishes | thickness | unit area |
| No. | (w/v) | (cm) | (cm) | (g/m²) |
| 1 | 0.5% | 0.6 | Not | Not |
| | | | determinable, | determinable |
| | | | individual | |
| | | | fragments | |
| 2 | 1% | 0.6 | Irregular | 59 |
| | | | thickness | |
| 3 | 2% | 0.6 | 0.6 | 140 |
| 4 | 3.5% | 0.6 | 0.6 | 240 |
| 5 | 5% | 0.6 | 0.6 | 340 |
| 6 | 7.5% | 0.6 | 0.6 | 527 |

2. Lee-White Clotting Test:

The Lee-White clotting test was used to investigate the hemostyptic properties of the following test specimens: 15% strength aqueous dextran aldehyde solution and dextran aldehyde nonwoven (produced from 2% strength aqueous solution).

Blood freshly withdrawn from a dog is placed in three test tubes into which ca. 0.5 g of the test substance was weighed. The tubes are stored at 37° C. Three minutes after withdrawal of the blood, the first tube is removed from the heating block, turned through 90° and replaced in the heating block. The procedure is repeated at intervals of 30 seconds, and the time taken for the blood to clot is measured. After the blood in the first tube has clotted, the next tube is tested. The Lee-White clotting time is defined as the time at which the blood in all three tubes has clotted. As a parallel control, the Lee-White clotting time of the blood without test substance is determined. The result is shown in graph form in FIG. 6 and shows, alongside the Lee-White clotting time for the respective test substance, the Lee-White clotting time of the control.

The dextran aldehyde solution and the dextran aldehyde nonwoven clearly shorten the Lee-White clotting time and thus show the expected hemostyptic action, the pure dextran aldehyde solution having a particularly strong influence on blood coagulation as a result of the higher concentration and more rapid distribution. With the dextran aldehyde nonwoven from the 2% strength aqueous dextran aldehyde solution, the blood clotting time was able to be reduced by ca. 33% compared to the control reaction without test substance.

3. Animal Test on the Pig (Liver) to Investigate the Efficacy of the Hemostyptic Nonwoven:

The efficacy of the hemostyptic nonwoven in staunching severe parenchymal bleeding was determined in a test carried out on the pig. The hemostyptic nonwoven was used to staunch and to treat liver resections and cross incisions.

a) Liver Resections

Before the resection, the blood supply to the liver was temporarily interrupted by the Pringle maneuver in which the vessels in the hepatoduodenal ligament are clamped. The vessels in the lobe of the liver to be resected were clamped with a parenchyma clamp, and the resection was then performed by monopolar cauterization (HF surgery) (FIG. 7).

The voluminous blood vessels were fixed by suture material and the wound surface was covered with a moist nonwoven. After release of the blood flow, the wound was sufficiently supplied and there was no secondary bleeding (FIG. 8).

b) Cross Incision

The liver surface was traumatized by means of cross incisions (3 cm long, 0.5 cm deep, FIG. 9). In contrast to the resections, the blood supply to the liver was not interrupted during the traumatization.

The nonwoven (lyophilized from 2% strength dextran aldehyde solution) was applied in the dry state to the incision wound and modeled onto the wound by light pressure by means of a moist compress. The bleeding stopped and the wound was sufficiently supplied (FIG. 10).

4. Synthesis of the Spacer Between Polymer and Aldehyde Function (FIG. 18)

The spacer is synthesized starting from an n-carboxy alkylaldehyde 17. To protect the aldehyde group, 17 is dissolved in an excess of ethylene glycol 18 and converted to the corresponding acetal 19 under reflux. The acetalization takes place in acid medium, and the catalyst used can be p-toluenesulfonic acid or 85% strength phosphoric acid. Because of the equilibrium reaction, the acetal, after binding the spacer to the polymer, can be converted back to the aldehyde. The acetals are stable in the neutral and alkaline. The conversion of the carboxy group to an active ester 21 takes place in anhydrous DMSO by means of dicyclohexylcarbodiimide (DCC) and N-hydroxysuccinimide 20 at room temperature and pH 7. An alternative possibility is conversion of the carboxy group with thionyl halides (SOCl₂ or SOBr₂) into an acid chloride.

5. Binding of the Spacer to the Polymer and Preparation of the Aldehyde Function (FIG. 19)

The binding of the spacer 21a in the form of an activated ester or an acid chloride to the polymer 22 is again carried out in dry DMSO with cleavage of N-hydroxysuccinimide or of the halide. The yield can be increased by addition of a base (e.g. pyridine). In the last step, ethylene glycol 18 is detached under acid conditions to the aldehyde group, and obtain the polymer with spacer and aldehyde function 24 regenerate.

6. Composition of a Hemostyptic with Cross-Linking Agent and Softener

Composition 1: C 0/70
70 ml solution: dextran aldehyde (DA), chitosan and glycerol
Molar ratio: aldehyde group (in the DA) to amino group (chitosan) 28:1
Molar ratio: aldehyde group (in the DA) to glycerol 10:1

1.17 g of DA (3.34% w/v) and 0.17 g of glycerol (0.49% w/v) are dissolved in 35 ml of bidistilled water. In parallel, 0.15 g (0.432% w/v) of Protasan UP 213 Cl® (FMC-BioPolymer AS Oslo, Norway, chitosan salt with chloride as counterion) is dissolved in 35 ml of bidistilled water. The two solutions are combined (70 ml), agitated, poured into lyophilization dishes (150×110 mm) and then lyophilized. The thickness of the nonwovens after lyophilization is 3.5 mm. To improve the elasticity, the nonwovens are pressed to a thickness of 0.3-0.4 mm by means of a pressing device.

7. Composition of a Hemostyptic with Cross-Linking Agent, Softener and Additive for Improving Absorption Power

Composition 2: C 5/70
70 ml solution: dextran aldehyde (DA), chitosan, glycerol and carboxymethylcellulose (CMC)
Molar ratio: aldehyde group (in the DA) to amino group (chitosan) 28:1
Molar ratio: aldehyde group (in the DA) to glycerol 10:1
Molar ratio: aldehyde group (in the DA) to monomer CMC unit 1000:5

1.17 g of DA (3.56% w/v) and 0.17 g of glycerol (0.52% w/v) are dissolved in 32.8 ml of bidistilled water. In parallel, 0.15 g (0.432% w/v) of Protasan UP 213 Cl® is dissolved in 35 ml of bidistilled water and 22 mg of CMC (1% w/v) are dissolved in 2.2 ml of bidistilled water. The solutions are combined (70 ml), agitated, poured into lyophilization dishes (150×110 mm) and then lyophilized. The thickness of the nonwovens after lyophilization is 3.5 mm. To improve the elasticity, the nonwovens are pressed to a thickness of 0.3-0.4 mm by means of a pressing device.

8. Properties and Use of the Nonwovens C 0/70 and C 5/70 in Animal Tests

The degree of swelling of the nonwovens is determined as follows:

The nonwovens are cut, in the pressed and unpressed state, to a size measuring 20×20 mm, their dry weight (Wₜᵣ) is determined, and they are immersed in 100 ml of Sörensen buffer solution (pH 7.4) for 5 seconds. After a dripping time of 10 seconds, the wet weight (W_{aq}) is determined. The degree of swelling can be calculated by the following formula.

$$Q [ \% ] = \frac{{W}_{aq} - {W}_{tr}}{{W}_{tr}} \times 100$$

TABLE 2
Degrees of swelling of nonwovens C 0/70 and
C 5/70 in the pressed and unpressed state

| Product | Degree of swelling |
|---|---|
| C 0/70 unpressed | 2901% |
| C 0/70 pressed | 1688% |
| C 5/70 unpressed | 4124% |
| C 5/70 pressed | 1946% |

CMC improves the absorption power of the nonwovens and therefore makes them easier to model during the operation. The degree of swelling in water decreases slightly as a result of the pressing, but at 1700-2000% it is still very high and is sufficient for hemostasis of heavily bleeding tissues.

The efficacy of the pressed nonwovens C 0/70 and C 5/70 was investigated on the basis of the staunching of diffuse parenchymal bleeding in rats and compared with the efficacy of Lyostypt® (collagen nonwovens B/Braun Aesculap Tuttlingen).

The traumatization of the liver was produced by means of a 1.5 cm long and 0.3 cm deep cross incision (FIG. 21). The polysaccharide nonwovens were applied in the dry state to the wound and were modeled onto the tissue with the aid of a compress soaked in physiological saline solution (FIG. 23). Lyostypt was (according to the directions for use) applied to the wound in the dry state and with manual pressure. All three test items adhered very well to the tissue and could not be moved by the operator (FIG. 23). A comparison of the time from application of the test item to staunching of the bleeding confirms (see Table 3) the high efficacy of the polysaccharide nonwovens.

All 27 animals (9 animals per product) survived the operation. All animals showed normal conditions at latest 4 days after the operation.

Findings on dissection after 7, 14 and 21 days show a good course of healing of the wound in all the animals. The test items were covering the trauma. With the polysaccharide nonwovens, in contrast to Lyostypt, there were no adhesions of the lobes of the liver, so that they also prevent undesired adhesions. After 14 days, vessel infiltrations into the polysaccharide nonwovens could be observed.

The macroscopic findings on dissection were confirmed by histopathology (FIG. 24). Just 7 days after the operation, connective tissue cell layers had migrated into the polysaccharide nonwovens. The wound margin had healed very well, and new liver regeneration tissue had already formed within the first 7 days.

TABLE 3
Comparison of times from application of the hemostyptics to
staunching of the bleeding (9 test animals per product)

| Product | Time to hemostasis |
|---|---|
| C 5/70 | 32 seconds |
| C 0/70 | 38 seconds |
| Lyostypt ® | 62 seconds |

1. A reabsorbable hemostyptic self-adhering to human or animal tissue and essentially consisting of at least one polymer which carries free aldehyde groups and whose aldehyde groups are able to react with nucleophilic groups of the tissue, the hemostyptic being present in solid, porous and absorbent form.

2. The hemostyptic as claimed in claim 1, characterized in that it is present in the form of a three-dimensional body, in particular a sheet.

3. The hemostyptic as claimed in claim 1, characterized in that it is present in the form of a nonwoven, in particular a three-dimensional nonwoven.

4. The hemostyptic as claimed in claim 1, characterized in that it is present in the form of an open-cell foam.

5. The hemostyptic as claimed in claim 1, characterized in that it is present in the form of a granulate or powder of absorbent particles.

6. The hemostyptic as claimed in claim 1, characterized in that the polymer, preferably the entire hemostyptic, is water-soluble.

7. The hemostyptic as claimed in claim 1, characterized in that the polymer carrying aldehyde groups is an oxidized, in particular bioabsorbable polysaccharide.

8. The hemostyptic as claimed in claim 7, characterized in that the oxidized polysaccharide is one from the group comprising starch, cellulose, agar, dextran, xanthan, heparin, hyaluronic acid, alginic acid and chrondoitin sulfate, preferably dextran polyaldehyde.

9. The hemostyptic as claimed in claim 1, characterized in that the proportion of glucose oxidized to the aldehyde in the dextran polyaldehyde is at least 20%, preferably 35% to 100%, in particular 60% to 80%.

10. The hemostyptic as claimed in claim 1, characterized in that the polymer carrying aldehyde groups is an in particular branched polyethylene glycol with at least 3 terminal aldehyde groups.

11. The hemostyptic as claimed in claim 1, characterized in that the polymer carrying aldehyde groups is an in particular branched polyvinyl alcohol with at least 3 terminal aldehyde groups.

12. The hemostyptic as claimed in claim 1, characterized in that it can be obtained by lyophilization of a solution of the at least one polymer.

13. The hemostyptic as claimed in claim 1, characterized in that it can be obtained from a 0.5-20% strength, preferably 1-15% strength, in particular 1-10% strength, especially 2% strength solution of the at least one polymer.

14. The hemostyptic as claimed in claim 1, characterized in that, because of its hydrophilic character and its porosity, it is able to take up at least 30 times its weight of fluid.

15. The hemostyptic as claimed in claim 1, characterized in that it is partially cross-linked with a cross-linking agent.

16. The hemostyptic as claimed in claim 15, characterized in that the cross-linking agent is at least one from the group comprising chitosan, bifunctional or multifunctional amines, bifunctional or multifunctional molecules with -AH and NH₂ groups, and bifunctional or multifunctional thiols, preferably chitosan.

17. The hemostyptic as claimed in claim 1, characterized in that it contains at least one additive for increasing the absorbency.

18. The hemostyptic as claimed in claim 17, characterized in that the agent for increasing the absorbency is carboxymethycellulose (CMC).

19. The hemostyptic as claimed in claim 1, characterized in that it has a surface structured at least on one side.

20. A method for producing a hemostyptic as claimed in claim 1, characterized in that at least one polymer in solution and/or in the gel state, preferably polysaccharide, in particular dextran polyaldehyde, is converted by means of lyophilization into a solid dry form.

21. Provision of the hemostyptic as claimed in claim 1, for a preferably internal application in an organism, in particular in wounds.

22. Provision of the hemostyptic as claimed in claim 1, for wound closure, preferably of internal wounds.

23. Provision of the hemostyptic as claimed in claim 1, for hemostasis in cases of organ resection or organ rupture.

24. Provision of the hemostyptic as claimed in claim 1, in the form of a ring for anastomoses.

25. Provision of a resorbable hemostyptic self-adhering to human or animal tissue and essentially consisting of at least one polymer which carries free aldehyde groups and whose aldehyde groups are able to react with amino groups of the tissue, the hemostyptic being present in a moist form, in particular a liquid or gel-like form.
